(19) United States
(12) Patent Application Publication
(10) Pub. No.: US 2007/0032657 A1
(43) Pub. Date: Feb. 8, 2007

(54) Method of preparation of nitroaminopyridine compounds

(76) Inventors: Prashant P. Deshpande, Princeton, NJ (US); John Anthony Grosso, Princeton Junction, NJ (US); Apurba Bhattacharya, Hyderabad (IN); Vikram C. Purohit, College Station, TX (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/492,730

(22) Filed: Jul. 25, 2006

Related U.S. Application Data
(60) Provisional application No. 60/705,261, filed on Aug. 3, 2005.

(51) Int. Cl. C07D 213/72

(52) U.S. Cl. 546/307

(57) ABSTRACT

A method of preparing an intermediate 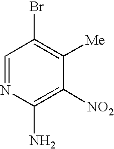 which is useful for the preparation of azaindole derivatives.

### CROSS-REFERENCE TO RELATED APPLICATIONS

This application claims the benefit of U.S. Provisional Application Ser. No. 60/705,261 filed Aug. 3, 2005.

### FIELD OF THE INVENTION

The present invention provides a method of preparing an intermediate useful for the preparation of azaindole derivatives.

### SUMMARY OF THE INVENTION

One aspect of the present invention provides a method of preparing a compound of formula 4: 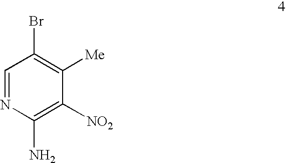
including the steps of:

(a) converting a compound of formula 1:

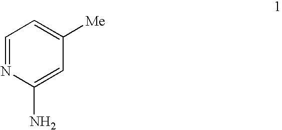

to a compound of formula 2:

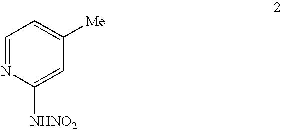

(b) brominating said compound of formula 2 to provide a compound of formula 3:

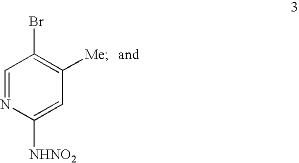

(c) converting said compound of formula 3 to provide said compound of formula 4.

A further aspect of the present invention provides a method of preparing a compound of formula 3:

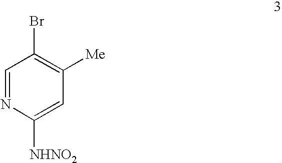

including the steps of:

(a) converting a compound of formula 1:

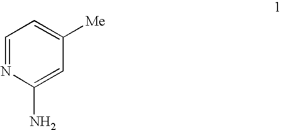

to a compound of formula 2:

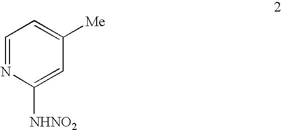

(b) brominating said compound of formula 2 to provide a compound of formula 3.

### DETAILED DESCRIPTION OF THE INVENTION

The present invention provides processes for the preparation of 2-amino,5-bromo, 3-nitropicoline, otherwise referred to herein as the compound of formula 4. This compound may be used, for example, as an intermediate useful in the preparation of azaindoles, such as those represented by the compound of formula 8. The compound of formula 8 is an azaindole derivative useful in the treatment of HIV. The present invention includes the preparation of the compound of formula 4 in addition to the preparation of intermediates as shown in the following scheme. Each of the steps are shown below in more detail following the general Scheme.

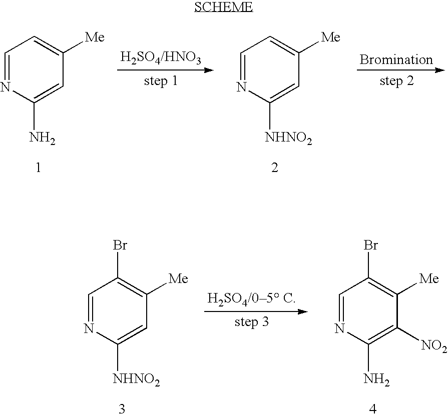

Step 1 includes the conversion of the compound of formula 1 to the compound of formula 2, but the introduction of a nitro function to the amine. This is conducted with a strong acid such as sulfuric acid (H₂SO₄) followed by nitric acid (HNO3).

Step 2 is the bromination of the compound of formula 2 to a compound of formula 3, which may be performed by a variety of reaction conditions. The reaction may be conducted either at room temperature or with heating. One of the following combinations may also be included (i) acetic acid and sodium acetate followed by introduction of bromine and acetic acid, (ii) dicholoro(m)ethane, acetonitrile and N-bromosuccinamide, (iii) acetic acid and potassium hydrogen phosphate followed by the introduction of bromine and acetic acid, and (iv) dichloromethane, water and tetrabutylammoniumtribromide.

Step 3 is the conversion from the compound of formula 3 to the compound of formula 4 is conducted with a strong acid such as sulfuiric acid.

The features and advantages of the present invention are more fully shown by the following examples which are provided for purposes of illustration, and are not to be construed as limiting the invention in any way.

### EXAMPLES

### Example 1—Step 1

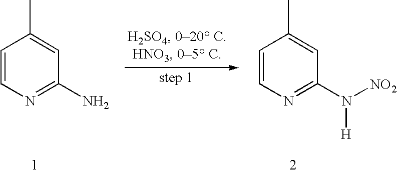

In a 500 mL 3-neck flask, equipped with a stirrer and an internal thermocouple probe (JKEM) was charged 116 mL of concentrated sulfuric acid (H₂SO₄) and allowed to cool between 0-5° C. with an ice bath. Then 25 g of the compound of formula 1 was charged, over a period of one hour, via an addition funnel.

After one hour, 15.5 mL (1 (eq.)) of concentrated nitric acid (69-71%) was added dropwise maintaining the reaction mixture between −3° to 0° C. with external cooling (ice/methanol) over a period of 1.25 hours. The mixture was allowed to stir between 0-3° C. for one hour, after which HPLC analysis revealed complete conversion of the compound of formula 1 to the compound of formula 2.

The solution was slowly quenched over 600 g of we ice in a 1 L beaker. The ice was allowed to melt and the resulting white slurry was stirred at room temperature for 10 min. The slurry was vacuum filtered, dried in vacuo (5 in Hg) at 50° C. (overnight) giving 84% recovered yield of the compound of formula 2.

Analytical data: ¹H-NMR (CD₃OD) (δ, ppm): 2.50 (s, 3H), 7.20 (bd, 1H, J=6.6 Hz, Ar—H), 7.30 (bs, 1H, Ar—H), 8.03 (d, 1H, J=6.6 Hz, Ar—H): ¹³C-NMR (CD₃OD) (δ, ppm): 21.00, 118.54, 119.91, 135.34, 144.00, 158.46: HRMS calcd for C₆H₇N₃O₂ 153.05383 found (M+1) 154.06166.

### Example 2 —Step 2a

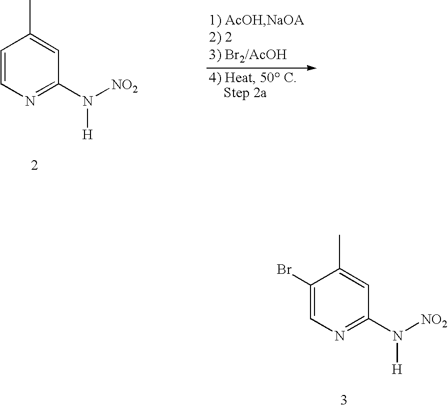

To a 50 mL single neck round bottom flask was charged 23 mL of acetic acid and 3.66 g (8 eq.) of anhydrous sodium acetate. The resulting mixture was heated to 40° C. until complete dissolution of sodium acetate was achieved.

One gram of the compound of formula 2 was added at 40° C. and the reaction was allowed to come to room temperature. A molecular bromine solution 1.005 g (0.34 mL/13 mL acetic acid) was slowly added over a period of 30 minutes with an addition funnel and heated to 50° C. for three hours.

The slurry containing the product was aged for 30 minutes at room temperature. The product was filtered in vacuo, washed with 5 mL cold water, 2 mL dichloromethane and dried under reduced pressure (8 in Hg, 55° C.) for one hour. The desired mono bromo derivative of formula 3 was obtained in 70% isolated yield (1.045 g).

Analytical data: ¹H-NMR (CD₃OD/CDCl₃) (δ, ppm): 2.39 (s, 3H, —CH3), 7.63 (s, 1H, Ar—H), 8.22 (s, 1H, Ar—H): ³C-NMR (DMF-D7) (δ, ppm): 19.46, 116.05, 116.44, 147.22, 147.80, 159.81: HRMS calcd for C₆H₆BrN₃O₂ 230.96434 found 231.97217.

### Example 3 —Step 2b

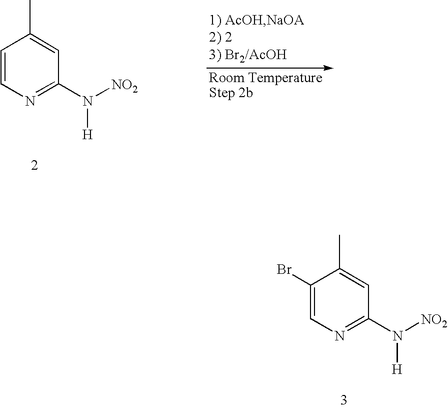

To a 250 mL single neck round bottom flask was charged 70 mL of acetic acid and 8.23 g (6 eq.) of anhydrous sodium acetate. The resulting mixture was stirred at 40° C. until complete dissolution of sodium acetate was achieved.

Three grams of the compound of formula 2 was added at 40° C. under high stirring. This slurry was allowed to come to room temperature (15 minutes). A molecular bromine solution, 31.14 g in 40 mL acetic acid, was slowly added over a period of 30 minutes with an addition funnel and the mixture allowed to stir at room temperature for 24 hours.

The slurry containing the product was aged for an additional 6 hours at room temperature. The product was filtered in vacuo, washed with 5 mL cold water, 2 mL dichloromethane and dried under reduced pressure (8 in Hg, 55° C.) for one hour. The desired crude mono bromo derivative of formula 3 was obtained in 83% isolated yield (3.75 g). See step 2a for analytical data.

### Example 4 —Step 2c

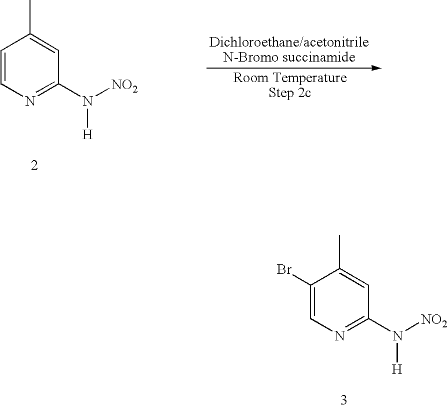

To a 250 mL single neck round bottom flask was charged 70 mL of 1,2-dichloroethane and 20 mL of acetonitrile. Then, 3.488 g of N-Bromo Succinamide (NBS) (1 eq.) was added at one time to this mixture and allowed to stir in the dark. Upon complete dissolution of the NBS, 3 g of the compound of formula 2 was added at one time. This white slurry was allowed to stir at room temperature for a period of 15 hours.

After 15 hours, the brown slurry was filtered in vacuo, washed with 3 mL dichloromethane and dried under reduced pressure (5in Hg, 50° C.) to yield 2.99 g of the crude product. See step 2a for analytical data.

### Example 5 —Step 2d

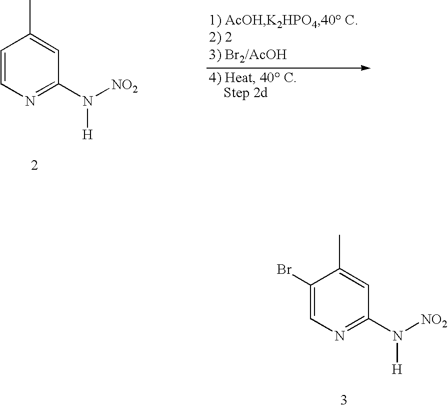

To a 50 mL single neck round bottom flask was charged 65 mL of acetic acid and 4.5 g (1 eq.) of potassium hydrogen phosphate di basic (K₂HPO₄). The resulting mixture was heated to 40° C. until complete dissolution of the potassium hydrogen phosphate di basic was achieved.

Then, 3 g of the compound of formula 2 was added at 40° C. and the reaction was allowed to come to room temperature. A molecular bromine solution, 3.137 g (5.025 mL/40 mL acetic acid) was slowly added over a period of 30 minutes with an addition funnel at room temperature and heated to 40° C. for 12 hours.

The slurry containing the product was aged for 30 min at room temperature. The product was filtered in vacuo, washed with 5 mL cold water, 2 mL dichloromethane and dried under reduced pressure (8 in Hg, 55° C.) for 3 hours to yield the desired mono bromo derivative of formula 3 in 65% isolated yield (corrected for purity, 2.867 g). See step 2a for analytical data.

### Example 6—Step 2e

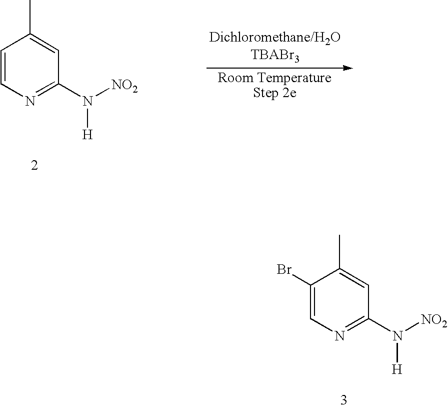

To a 50 mL single neck round bottom flask was charged 30 mL of dichloromethane and 3.15 g (1.3 eq.) of tetrabutylammoniumtribromide. Then, 1 g of the compound of formula 2 was added at one time under stirring. After 10 minutes, 20 mL of water was introduced under high stirring. The biphasic reaction mixture was allowed to stir for 48 hours, and aged for an additional one hour at room temperature. The desired mono bromo derivative was filtered, washed with 1 mL of dichloromethane and dried in vacuo (5 in Hg, 50° C., 2 hours) to yield 01.02 g of the compound of formula 3 (65%, based on purity of the isolated solid). See step 2a for analytical data.

### Example 7—Step 3

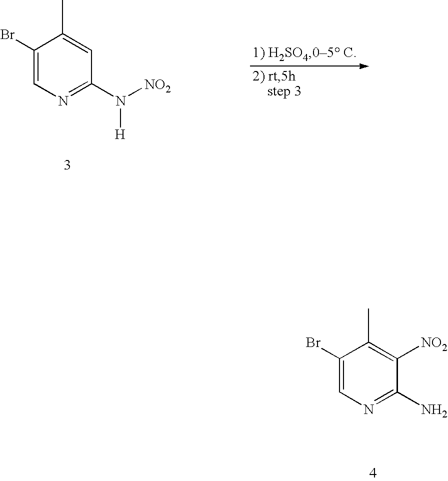

To a 50 mL single neck flask equipped with a stirrer, an external cooling, and an internal thermocouple probe, was added 10 mL of concentrated sulfuric acid. The mixture was stirred between 0-5° C. with an external ice bath. Then, 1 g of the compound of formula 3 was added slowly to the solution. After one hour, the reaction was allowed to come to room temperature and stirred for an additional 5 hours.

The reaction was quenched over 50 g of wet ice and neutralized with 50% NaOH solution to pH 6.0 under constant stirring.

The desired compound of formula 4 was obtained as yellow slurry which was filtered and dried in vacuo (5 in Hg, 50° C.) for 3 hours. The product of formula 4 was recovered in 98% yield (0.98 g).

Analytical data: m.p. 132° C. IR (KBr, cm⁻¹): 1633, 1581, 1538, 1512, 1458, 1377, 1344, 1321, 1244, 869, 779. ¹H-NMR (CDCl₃) (δ, ppm): 2.55 (s, 3H), 5.85 (bs, 2H), 8.25 (s, 1H): ¹³C-NMR (CDCl₃) (δ, ppm): 20.81, 112.14, 144.49, 151.91, 153.78 (2C); HRMS: calcd for C₆H₆BrN₃O₂: 230.96434; found: (M+1): 231.97217.

### Example 8 —Step 3

The procedure in Example 7 was repeated using 72 mL of sulfuric acid which was maintained between 0-5° C. Then, 11.28 g of the compound of formula 3 was added over a period of 20 minutes and stirred for an additional 20 minutes. The reaction mixture was allowed to come to room temperature and stirred for an additional one hour.

The reaction was quenched over 300 of wet ice and aged for 30 minutes. This cold solution was neutralized with 200 ml of 50% sodium hydroxide to pH 5.2. The resulting yellow slurry was filtered and dried in vacuo (5 in Hg, 40° C., 10 hours) to yield 9.53 g of the compound of formula 4 as a yellow solid in 93% recovery. See Example 7 for analytical data.

While there have been described what are presently believed to be the preferred embodiments of the invention, those skilled in the art will realize that changes and modifications may be made thereto without departing from the spirit of the invention, and it is intended to include all such changes and modifications as fall within the true scope of the invention.

What is claimed is:

1. A method of preparing a compound of formula 4: 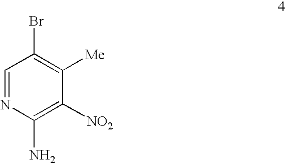
comprising the steps of:
(a) converting a compound of formula 1: 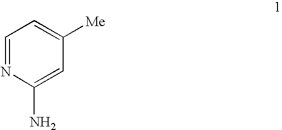
to a compound of formula 2: 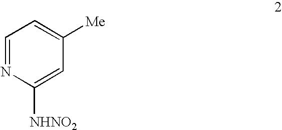
(b) brominating said compound of formula 2 to provide a compound of formula 3: 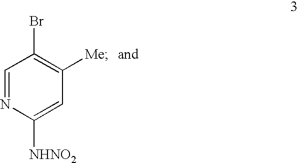
(c) converting said compound of formula 3 to provide said compound of 15 formula 4.

2. The method of claim 1, wherein said converting said compound of formula 1 to said compound of formula 2 is conducted by reaction with sulfuric acid followed by the introduction of nitric acid.

3. The method of claim 1, wherein said brominating said compound of formula 2 is conducted at room temperature.

4. The method of claim 1, wherein said brominating said compound of formula 2 is conducted with heating.

5. The method of claim 3, wherein said brominating further comprises steps of combining the compound of formula 2 with acetic acid and sodium acetate followed by introduction of bromine and acetic acid.

6. The method of claim 4, wherein said brominating further comprises steps of combining the compound of formula 2 with acetic acid and sodium acetate followed by introduction of bromine and acetic acid.

7. The method of claim 3, wherein said brominating is conducted in the presence of dichloroethane, acetonitrile and N-bromosuccinamide.

8. The method of claim 3, wherein said brominating further comprises steps of combining the compound of formula 2 with acetic acid and potassium hydrogen phosphate followed by introduction of bromine and acetic acid.

9. The method of claim 4, wherein said brominating is conducted in the presence of dichloromethane, water, and tetrabutylammoniumtribromide.

10. The method of claim 1, wherein said converting said compound of formula 3 to said compound of formula 4 is conducted in the presence of sulfuric acid.

11. A method of preparing a compound of formula 3: 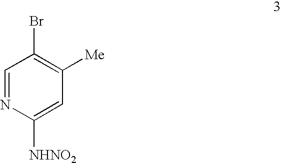
comprising the steps of:
(a) converting a compound of formula 1: 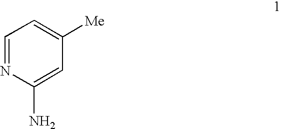
to a compound of formula 2: 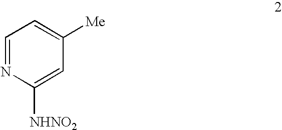
(b) brominating said compound of formula 2 to provide a compound of formula 3.
